(19) United States
(12) Patent Application Publication
(10) Pub. No.: US 2025/0342961 A1
(43) Pub. Date: Nov. 6, 2025

(54) METHOD AND DEVICE FOR DETERMINING ONSET OF SEPSIS IN EMERGENCY SET-UPS

(71) Applicant: Siemens Healthcare Diagnostics Inc., Tarrytown, NY (US)

(72) Inventors: Ankit GUPTA, Bangalore (IN); Ruchi CHAUHAN, Bhopal (IN)

(73) Assignee: Siemens Healthcare Diagnostics Inc., Tarrytown, NY (US)

(21) Appl. No.: 19/126,355

(22) PCT Filed: Oct. 31, 2023
(86) PCT No.: PCT/US2023/036346

§ 371 Date: May 1, 2025

(30) Foreign Application Priority Data
Nov. 4, 2022 (IN) 202241063134

(51) Int. Cl. G16H 50/20; A61B 5/00; G16H 50/30; G16H 50/70

(52) U.S. Cl. CPC G16H 50/20; A61B 5/7275; G16H 50/30; G16H 50/70

(57) ABSTRACT

A method and a device for determining onset of sepsis are provided. In one aspect, the method includes receiving a medical dataset associated with the patient. Further, the method includes determining if the plurality of medical parameters includes at least one sepsis specific parameter. Additionally, the method includes determining a first output parameter if the plurality of medical parameters does not include at least one sepsis specific parameter. The method also includes determining a second output parameter indicative of onset of sepsis in the patient if the plurality of medical parameters include at least one sepsis specific parameter, wherein the medical parameters associated with the patient are obtained for at least one time instance.

The present application is a national phase application of International Application No. PCT/US2023/036346 which claims the benefit of Indian Patent Application No. 202241063134, filed Nov. 4, 2022, the entire contents of each of which is incorporated by reference.

### TECHNICAL FIELD

The present disclosure relates to a method and a device for determining an onset of sepsis in a patient in emergency set-ups.

### BACKGROUND

Sepsis is an extreme immune response of an individual's body to an infection. Sepsis can be life-threatening, triggering a series of reactions throughout the body that may cause tissue damage, organ failure, and death. Early detection of sepsis is considered as one of the key aspects to improve the outcomes of sepsis treatments. Clinical criteria that assist in sepsis recognition are widely available, however the fundamental need for early detection and treatment of sepsis remains unmet. For about 30% to 50% of patients, sepsis treatment is initiated in the emergency department. Since the capacity of intensive care units (ICUs) is limited and since not all patients may benefit from ICU admission, it is a challenge to effectively stratify patients into ones that may require treatment in an ICU and ones who can be treated in the emergency department. Incorrect stratification may cause increased morbidity and mortality and increased length of stay in hospitals.

Currently, there is no way to accurately stratify patients as ones who require treatment in an ICU and ones who can be treated in an emergency department, especially due to limited amount of patient health information available in an emergency department. Therefore, there is a need for a method and device that enables timely determination of onset of sepsis in a patient with limited patient dataset, and is effective and accurate.

### SUMMARY AND DESCRIPTION

The object of the disclosure is therefore to provide a method and a device that enables effective determination of onset of sepsis in a patient with limited patient dataset.

The disclosure achieves the object by a method of determining onset of sepsis in a patient. The method comprises receiving a medical dataset associated with the patient. The medical dataset includes, for example, a plurality of medical parameters associated with the patient. The medical parameters are a representation of a medical condition of the patient. In an embodiment, the medical dataset may be stored in a medical database and may be accessed when needed. The medical parameters may include parameters that are available in an emergency department. The method further includes determining if the plurality of the medical parameters includes at least one sepsis specific parameter. Further, if it is determined that the plurality of medical parameters does not include at least one sepsis specific parameter, the medical parameters are provided to a first trained machine learning model based on which a first output parameter is determined. In an embodiment, the first output parameter is a preliminary indication of onset of sepsis in the patient. The first output parameter is derived from the plurality of medical parameters without the presence of a sepsis specific parameter. In a further embodiment, the first output parameter is indicative of whether at least one sepsis specific parameter is to be obtained. In an embodiment, the medical parameters associated with the patient are obtained for at least one time instance.

The method further includes determining a second output parameter by a second trained machine learning model if the plurality of medical parameters associated with the patient includes at least one sepsis specific parameter. The second output parameter is indicative of onset of sepsis in the patient. Advantageously, the method enables determination of onset of sepsis in a patient based on at least one time instance of medical information. Therefore, the method does not rely on continuous medical information associated with the patient, which makes it easier to deploy the present disclosure in an emergency department.

According to an embodiment, the plurality of medical parameters includes a combination of laboratory parameters and sepsis specific parameters. For example, the medical parameters include, but are not limited to, blood urea nitrogen, creatinine, bilirubin, white blood cells, platelets, lactate/lactic acid, C-reactive protein (CRP), procalcitonin, interleukin-6 (IL-6), or combinations thereof. In a further embodiment, availability of at least four parameters of the above parameters enables determination of onset of sepsis in the patient. An advantage of the disclosure is that it relies on only a few medical parameters to determine the onset of sepsis in a patient. Further, the accuracy of the second output parameter is increased if sepsis specific parameters are provided to the second trained machine learning model.

According to an embodiment, the first output parameter is a risk score, wherein if the risk score is below a first pre-defined threshold, it is an indication of no onset of sepsis in the patient. For example, the first pre-defined threshold is 0.2. Similarly, if the risk score is above a second pre-defined threshold, it is an indication of onset of sepsis in the patient. For example, the second pre-defined threshold is 0.8. Further, if the risk score is within a third pre-defined threshold range, it is an indication of obtaining at least one sepsis specific parameter associated with the patient. For example, the third pre-defined threshold range associated with the first output parameter is between 0.2 and 0.8. Therefore, a risk score that may fall in the range between 0.2 and 0.8 may indicate a need to obtain at least one sepsis specific parameter associated with the patient such that confidence of prediction of onset of sepsis in the patient is improved. Advantageously, the at least one sepsis specific parameter may be obtained only if an indication of its need is determined by the trained first machine learning model. This enables the patient to undergo testing for sepsis specific parameters only if there exists a need for it.

Advantageously, the method enables determination of an onset of sepsis in the patient even if the medical parameters do not include a sepsis specific parameter. This enables the disclosure to be deployed in an emergency department where availability of sepsis specific parameter data associated with the patient may be limited.

According to another embodiment, the second output parameter indicative of onset of sepsis in the patient is a risk score. Therefore, if the risk score is above a fourth pre-defined threshold, it is an indication of onset of sepsis. For example, the fourth pre-defined threshold is 0.5. Therefore, a risk score above 0.5 is an indication of onset of sepsis in a patient. Advantageously, the determination of onset of sepsis is enabled with minimal set of medical parameters associated with the patient.

According to an embodiment, the first trained machine learning model and the second trained machine learning model are Extreme Gradient (XG) Boost classification models. XGBoost is a supervised learning algorithm that is used for regression and classification of large datasets. XGBoost model uses a sequentially built shallow decision trees to provide accurate results and a highly scalable training method that avoids overfitting. Decision trees are created in a sequential form and weights are assigned to all independent variables. The decision tree predicts the results based on the weights assigned to the independent variables. If the variables are predicted wrong by a decision tree, the weight associated with such variables is increased. These variables are then fed to a second decision tree. In particular, the individual classifiers ensemble to provide a more accurate and precise model. Advantageously, XGBoost model enables accurate prediction of onset of sepsis in a patient, especially with limited medical parameters.

According to yet another embodiment, the method further includes determining a root cause of the risk score determined by the first trained machine learning model. Similarly, a root cause of the risk score determined by the second trained machine learning model may also be determined. For example, the first trained machine learning model may use SHapley Additive exPlanations (SHAP) library to give feature importance. SHAP is a game theoretic approach to explain an output of the machine learning model. Higher SHAP values indicate a greater contribution to the prediction of sepsis and vice versa. Advantageously, determining the root cause associated with the with risk score enables availability of additional medical insights to the clinicians. This ensures that the patient receives a more precise treatment.

The method also achieves the object by a method of training the first machine learning model and the second machine learning model. The method includes receiving a medical dataset associated with the patient. The medical dataset includes a plurality of medical parameters associated with the patient. The medical dataset may be received from a source such as medical database. The plurality of medical parameters is extracted from the medical dataset. The parameters may reflect a medical condition of the patient in real-time or near real-time. The method further includes determining if the plurality of medical parameters includes at least one sepsis specific parameter. For example, the sepsis specific parameter may include lactic acid/lactate, C-reactive protein (CRP), procalcitonin, and/or IL-6.

Further, the first machine learning model and the second machine learning model are received. If the plurality of the medical parameters associated with the patient does not include at least one sepsis specific parameter, the first machine learning model determines a first output parameter. The first output parameter is a preliminary indication of an onset of sepsis in the patient. The first output parameter is determined on the basis on the medical parameters in the medical dataset, without considering the sepsis specific parameters. Additionally, the first output parameter is also indicative of a need for obtaining at least one sepsis specific parameter associated with the patient. In an embodiment, the sepsis specific parameters may improve the accuracy with which the onset of sepsis in a patient is determined.

The method further includes receiving sepsis data related to the medical dataset, wherein the sepsis data indicates an onset of sepsis or indicates no presence of sepsis at a defined time period in the patient associated with the medical dataset. The sepsis data may pertain to the patient/a plurality of patients that captures the actual medical outcome associated with the patient. Therefore, the sepsis data is an indication of whether the patient actually had sepsis or not. In an embodiment, the sepsis dataset may include a medical dataset that has been labelled to be indicative of onset of sepsis or no presence of sepsis for a defined time period in a patient. In a further embodiment, the labelled medical dataset may be associated with a plurality of patients historically monitored and treated for sepsis. The labelled medical dataset may include one or more features recorded at regular time intervals, thereby showcasing variation in the values associated with the one or more features over the time intervals. In an alternate embodiment, the sepsis data may be data received from a physician/expert that may include an analysis of one or more features present in the medical dataset associated with the patient, indicating the onset of sepsis or presence of no sepsis. The sepsis data is therefore used for comparing the output of the first machine learning model with the actual sepsis data associated with the patient.

In a further step, the first machine learning model is adjusted based on the outcome of the comparison between the first output parameter and the sepsis data. Advantageously, the adjusting the first machine learning model based on the sepsis data enables improving the accuracy of the first machine learning model. Therefore, the determination of the first output parameter is performed with greater accuracy.

Additionally, if the plurality of medical parameters includes at least one sepsis specific parameter, the second machine learning model determines a second output parameter. The second output parameter is an indication of onset of sepsis in the patient. In an embodiment, the accuracy of the second output parameter is greater over the first output parameter since the second output parameter is determined using the sepsis specific parameters associated with the patient in addition to the other medical parameters. Further, the second output parameter is compared with the sepsis data associated with the patient and the second machine learning model is adjusted based on an outcome of the comparison. The second machine learning model may be adjusted if a difference between the second output parameter and the sepsis data is identified in the comparison. Advantageously, the machine learning model is made more robust, thereby improving the accuracy with which the second output parameter is determined by the model. Therefore, determination of the onset of sepsis in the patient may be made effective and timely.

According to an embodiment, the method further includes pre-processing the medical dataset. In pre-processing the medical dataset, the method includes imputing at least one missing value associated with the plurality of medical parameters in the medical dataset. In particular, a missing value associated with the features in the medical dataset is identified. In an embodiment, a value associated with one or more features in the medical dataset may be missing if the values may not have been captured/recorded for the patient, at a given time interval. Alternatively, the value associated with the one or more features in the medical dataset may be considered as missing if the values are unplausible or beyond reasonable thresholds. Accordingly, a value preceding the missing value associated with the features is identified. The value preceding the missing value may be the value last captured/recorded for the patient before the given time interval. The missing value is substituted with the value preceding the missing value associated with the features in the medical dataset. In an embodiment, the substitution of the missing value with the preceding value may be performed for a limited time period within which the values are recorded. The time period may be dependent on a type of the one or more features in the medical dataset. For example, the time period defined for laboratory parameters is at a range of 22 to 24 hours. The values may be finalized based on data statistics and clinicians' input. Advantageously, the medical dataset is made complete and more usable by the first machine learning model and the second machine learning model. Therefore, the accuracy of determination of the onset of sepsis in the patient is improved.

Further, the method includes sampling the plurality of medical parameters based on a time instance associated with each of the medical parameters in the plurality of medical parameters. Sampling of medical parameters is performed to capture most meaningful medical information from the plurality of medical parameters. Sampling the medical parameters enables application of the present disclosure in an emergency department-based set-up where patient data may not be available for a time-series.

In an embodiment, sampling of the plurality of medical parameters comprises identifying a pre-onset and a post-onset time period from the sepsis data related to the medical dataset, wherein the medical dataset is associated with a patient identified as sepsis positive. For example, the pre-onset and post-onset time period may be in the range of 5 to 8 hours before and after the onset of sepsis in the patient and more specifically in the range of 4 to 6 hours before and after the onset of sepsis in the patient. At least one data point from the sepsis data is extracted from an earliest time instance in the pre-onset time period and/or the post-onset time period. The data point corresponds to the plurality of medical parameters associated with the patient. The earliest time instance may be, for example, a first time instance for which data point is available in the pre-onset and post-onset time period.

In a further embodiment, sampling the plurality of medical parameters further includes extracting at least one data point from the sepsis data related to the medical dataset, wherein the medical dataset is associated with a patient identified as sepsis negative. Median of the onset times is used for sampling data points associated with patients identified as sepsis negative. Further, data points from patients identified as sepsis negative are under-sampled to match the number of data points received from patients identified as sepsis positive. Since two data points are considered from sepsis positive patients (one data point each for pre-onset and post-onset times) and one data point is considered from sepsis negative patients, the patient data from which negative data points are considered may be picked twice as that of patient data from which positive data points are considered. Advantageously, sampling data points enables the disclosure to work in an emergency department set-up. Since an emergency department may have access to only a single value of a given medical parameter and a time-series information may not be available, sampling the data points trains the machine learning models to function in presence of limited data points. Thus, a need for time-series data to determine an onset of sepsis in a patient is eliminated.

The object of the disclosure is also achieved by a sepsis determination device for determining an onset of sepsis in a patient. The device comprises one or more processing units, a medical database coupled to the one or more processing units, the medical database comprising a plurality of medical datasets associated with the patient and sepsis data. The device further comprises a memory coupled to the one or more processing units. The memory comprises a sepsis determination module configured to perform the method steps as described above, using a first trained machine learning model and a second trained machine learning model.

The disclosure relates in one aspect to a computer program product comprising a computer program, the computer program being loadable into a storage unit of a system, including program code sections to make the system execute the method according to an aspect when the computer program is executed in the system.

The disclosure relates in one aspect to a computer-readable medium, on which program code sections of a computer program are saved, the program code sections being loadable into and/or executable in a system to make the system execute the method according to an aspect when the program code sections are executed in the system.

The realization of the disclosure by a computer program product and/or a computer-readable medium has the advantage that already existing management systems can be easily adopted by software updates in order to work as proposed by the disclosure.

The computer program product can be, for example, a computer program or comprise another element apart from the computer program. This other element can be hardware, (e.g., a memory device), on which the computer program is stored, a hardware key for using the computer program and the like, and/or software, (e.g., a documentation or a software key for using the computer program).

### BRIEF DESCRIPTION OF THE DRAWINGS

The present disclosure is further described hereinafter with reference to illustrated embodiments shown in the accompanying drawings, in which:

FIG. 1 illustrates a block diagram of a sepsis determination device in which an embodiment for determining onset of sepsis in a patient can be implemented.

FIG. 2 illustrates a flowchart of a method of determining the onset of sepsis in a patient, according to an embodiment.

FIG. 3 illustrates a flowchart of a method of training a machine learning model for determining the onset of sepsis in the patient, according to an embodiment.

FIG. 4 illustrates a flowchart of a method of pre-processing the medical dataset, according to an embodiment.

FIG. 5 illustrates a graphical representation of sampling medical parameters, according to an embodiment.

FIG. 6 illustrates a working of the machine learning model for determining the onset of sepsis in the patient, according to an embodiment.

FIG. 7 illustrates a graphical representation of root cause analysis of risk score determined by the first and second trained machine learning models, according to an embodiment.

### DETAILED DESCRIPTION

Hereinafter, embodiments for carrying out the present disclosure are described in detail. The various embodiments are described with reference to the drawings, wherein like reference numerals are used to refer to like elements throughout. In the following description, for purpose of explanation, numerous specific details are set forth in order to provide a thorough understanding of one or more embodiments. It may be evident that such embodiments may be practiced without these specific details.

In the following, the solution according to the disclosure is described with respect to the claimed providing systems as well as with respect to the claimed methods. Features, advantages, or alternative embodiments herein can be assigned to the other claimed objects and vice versa. In other words, claims for the providing systems can be improved with features described or claimed in the context of the methods. In this case, the functional features of the method are embodied by objective units of the providing system.

Furthermore, in the following, the solution is described with respect to methods and systems for determining an onset of sepsis in a patient as well as with respect to methods and systems for training a machine learning model for determining an onset of sepsis in a patient. Features, advantages, or alternative embodiments herein can be assigned to the other claimed objects and vice versa. In other words, claims for methods and systems for training the machine learning model for determining an onset of sepsis in a patient can be improved with features described or claimed in context of the methods and systems for determining an onset of sepsis in a patient, and vice versa. In particular, the trained machine learning model of the methods and systems for determining an onset of sepsis in a patient can be adapted by the methods and systems for training the machine learning model for determining an onset of sepsis in a patient. Furthermore, the input data can comprise advantageous features and embodiments of the training input data, and vice versa. Furthermore, the output data can comprise advantageous features and embodiments of the output training data, and vice versa.

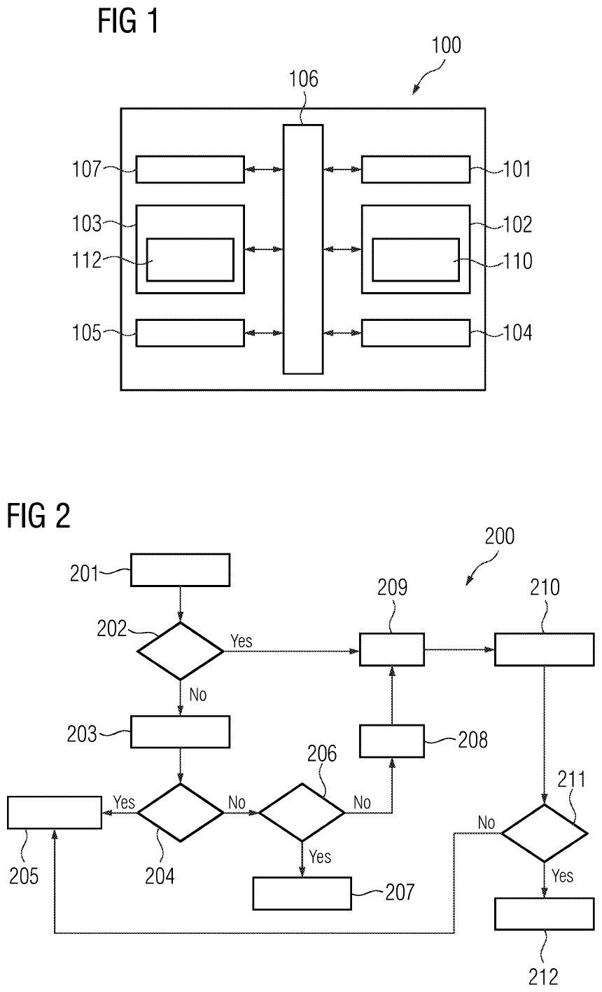

FIG. 1 is a block diagram of a sepsis determination device 100 in which an embodiment can be implemented, for example, as a device 100 for determining an onset of sepsis in a patient, configured to perform the processes as described therein. In FIG. 1, the device 100 comprises a processing unit 101, a memory 102, a storage unit 103, an input unit 104, a bus 106, an output unit 105, and a network interface 107.

The processing unit 101, as used herein, means any type of computational circuit, such as, but not limited to, a microprocessor, microcontroller, complex instruction set computing microprocessor, reduced instruction set computing microprocessor, very long instruction word microprocessor, explicitly parallel instruction computing microprocessor, graphics processor, digital signal processor, or any other type of processing circuit. The processing unit 101 may also include embedded controllers, such as generic or programmable logic devices or arrays, application specific integrated circuits, single-chip computers, and the like.

The memory 102 may be volatile memory and non-volatile memory. The memory 102 may be coupled for communication with the processing unit 101. The processing unit 101 may execute instructions and/or code stored in the memory 102. A variety of computer-readable storage media may be stored in and accessed from the memory 102. The memory 102 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 102 includes a sepsis determination module 110 stored in the form of machine-readable instructions on any of the above-mentioned storage media and may be in communication to and executed by processor 101. When executed by the processor 101, the sepsis determination module 110 causes the processor 101 to process a medical dataset to determine an onset of sepsis in a patient. Method steps executed by the processor 101 to achieve the abovementioned functionality are elaborated upon in detail in FIGS. 2, 3, 4, 5 and 6.

The storage unit 103 may be a non-transitory storage medium that stores a medical database 112. The medical database 112 is a repository of medical dataset and sepsis data related to one or more patients that is maintained by a healthcare service provider. The input unit 104 may include input means such as keypad, touch-sensitive display, camera (such as a camera receiving gesture-based inputs), etc. capable of receiving input signal such as a medical image. The bus 106 acts as interconnect between the processor 101, the memory 102, the storage unit 103, the input unit 104, the output unit 105 and the network interface 107.

Those of ordinary skilled in the art will appreciate that the hardware depicted in FIG. 1 may vary for particular implementations. For example, other peripheral devices such as an optical disk drive and the like, Local Area Network (LAN)/Wide Area Network (WAN)/Wireless (e.g., Wi-Fi) adapter, graphics adapter, disk controller, input/output (I/O) adapter also may be used in addition or in place of the hardware depicted. The depicted example is provided for the purpose of explanation only and is not meant to imply architectural limitations with respect to the present disclosure.

A device 100 in accordance with an embodiment of the present disclosure includes an operating system employing a graphical user interface. The operating system permits multiple display windows to be presented in the graphical user interface simultaneously with each display window providing an interface to a different application or to a different instance of the same application. A cursor in the graphical user interface may be manipulated by a user through a pointing device. The position of the cursor may be changed and/or an event such as clicking a mouse button, generated to actuate a desired response.

One of various commercial operating systems, such as a version of Microsoft Windows™, a product of Microsoft Corporation located in Redmond, Washington may be employed if suitably modified. The operating system is modified or created in accordance with the present disclosure as described.

Disclosed embodiments provide systems and methods for processing medical dataset. In particular, the systems and methods may enable determination of an onset of sepsis in a patient.

FIG. 2 illustrates a flowchart of a method 200 of determining an onset of sepsis in a patient, according to an embodiment. At step 201, medical dataset associated with the patient is received from a source. In the present embodiment, the source is the medical database 112. The medical dataset includes a plurality of medical parameters associated with the patient. The medical parameters are, for example, laboratory parameters such as, but not limited to, blood urea nitrogen, creatinine, lactate/lactic acid, bilirubin total, white blood cells, blood platelets, C-reactive protein (CRP), procalcitonin, IL-6, etc. The medical parameters are chosen such that they are commonly available in an emergency department set-up. Therefore, values associated with the medical parameters may be available/recorded only at a single time instances.

Further, at step 202, it is determined if the medical parameters include at least one sepsis specific parameter. In particular, sepsis specific parameters are conventional markers used for diagnosis of sepsis. They may include IL-6, procalcitonin, lactic acid/lactate, and/or CRP. Therefore, depending upon the presence of sepsis specific parameters in the medical dataset, the method may provide an analysis of onset of sepsis in the patient. If sepsis specific parameters are not present in the medical dataset associated with the patient, at step 203, a first output parameter is determined using a first trained machine learning model. The first output parameter is a preliminary indication of onset of sepsis that is determined based on the other medical parameters present in the medical dataset, barring the sepsis specific parameters. In an embodiment, the first output parameter is a risk score that provides information on a risk of onset of sepsis in the patient.

The first output parameter is also an indication of whether at least one sepsis specific parameter associated with the patient is to be obtained. In an embodiment, sepsis specific parameter may substantiate the finding of the first trained machine learning model on whether there is an onset of sepsis in the patient.

At step 204, the first output parameter is compared with a first pre-defined threshold to determine if the first output parameter is equal to or below the first pre-defined threshold. The first pre-defined threshold may be a value of 0.2. If the first output parameter is equal to or below the first pre-defined threshold, at step 205, it is determined that there is no onset of sepsis in the patient. Otherwise, at step 206, it is determined if the first output parameter is equal to or greater than a second pre-defined threshold. The second pre-defined threshold may be a value of 0.8. If the first output parameter is equal to or above the second pre-defined threshold, at step 207, it is determined that there is onset of sepsis in the patient. Otherwise, it is determined that the first output parameter is within a third pre-defined threshold range. For example, the range may be between 0.2 and 0.8. If the first output parameter is within the third pre-defined threshold range, at step 208, it is determined if at least one sepsis specific parameter associated with the patient is to be obtained. An output to this effect may be provided to the user.

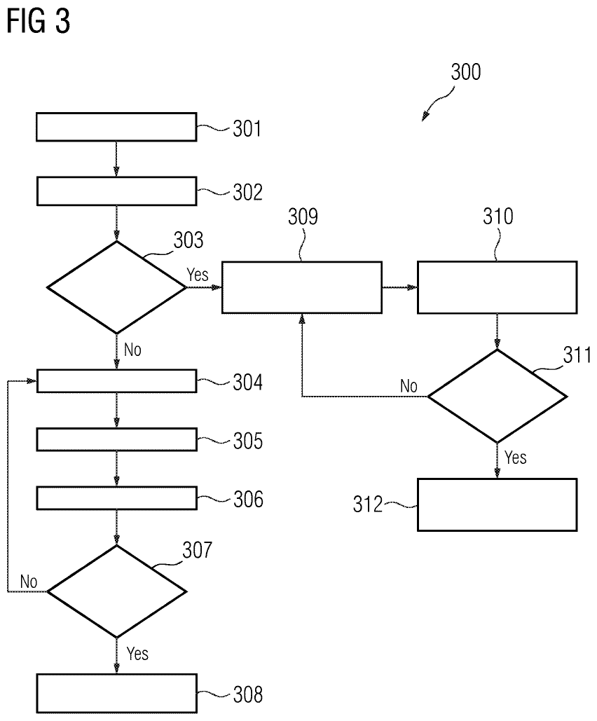

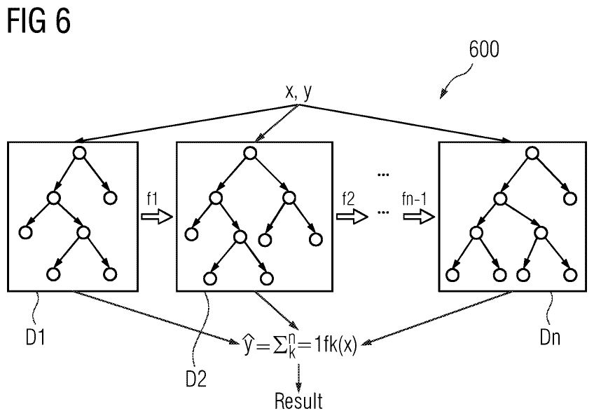

In an instance if it is determined at step 202 that at least one sepsis specific parameter exists in the medical dataset, at step 209, a second output parameter is determined by a second trained machine learning model. The second trained machine learning model analyses the medical parameters along with the sepsis specific parameter(s) to determine if there is an onset of sepsis in the patient. The second output parameter is a risk score in a range of 0.5 to 1. In an embodiment, if the at least sepsis specific parameter is obtained after step 208, the sepsis specific parameter is provided to the second trained machine learning model to determine the second output parameter at step 209. In the present embodiment, the first trained machine learning model and the second trained machine learning model are Extreme Gradient (XG) Boost classification model. The training and working of the machine learning model are described in further detail in FIGS. 3 and 6 respectively.

Further, at step 210, the second output parameter is compared with a fourth pre-defined threshold. The fourth pre-defined threshold is a risk score and is set at 0.5. At step 211, it is determined if the second output parameter is greater than the fourth pre-defined threshold. If yes, at step 212, it is determined that there is an onset of sepsis in the patient. In an embodiment, a notification may be output to the user indicating the onset of sepsis in the patient. For example, the notification in the form of a message/alert on a graphical user interface of the output unit 105. Alternatively, the notification may also be a sound-based alert to the user. If the second output parameter is not above the second pre-defined threshold, at step 205, an output is provided to a user as an indication of absence of sepsis in the patient.

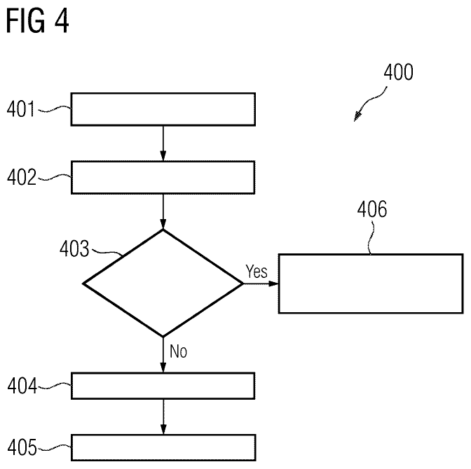

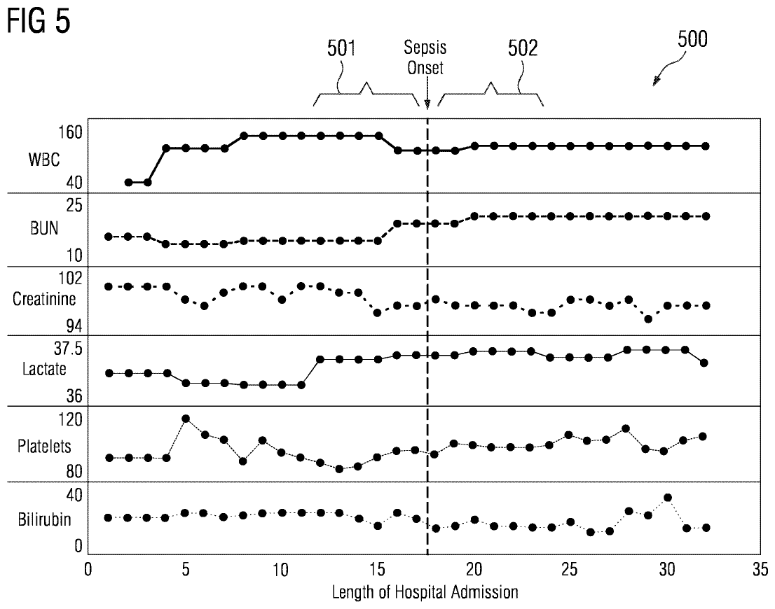

FIG. 3 illustrates a flowchart of a method 300 of training the first machine learning model and the second machine learning model for determining the onset of sepsis in the patient, according to an embodiment. At step 301, medical dataset associated with the patient is received. In an embodiment, the medical dataset is obtained from the medical database 112. In an embodiment, the medical dataset is pre-processed to ensure the right dataset is provided to the first machine learning model and the second machine learning model for training purposes. The method steps associated with pre-processing the medical dataset is elaborated in further detail in FIG. 4. The medical dataset includes a plurality of medical parameters associated with the patient, which enable determination of onset of sepsis in a patient. In a further embodiment, the medical dataset may include medical parameter values for at least one time instance. At step 302, the plurality of medical parameters is extracted from the medical dataset. Each of the medical parameters in the medical dataset has a value associated with it based on the patient condition. Therefore, the extracted medical parameters also include the value associated with the medical parameters.

Further, at step 303, it is determined if the extracted medical parameters include at least one sepsis specific parameter. If the medical parameters do not include any of the sepsis specific parameters, at step 304, the first machine learning model is used to determine a first output parameter. The first output parameter is a risk score that is a preliminary indication of onset of sepsis in the patient. The risk score also indicates a need to obtain at least one sepsis specific parameter associated with the patient. At step 305, sepsis data associated with the patient is received. The sepsis data is pre-defined information associated with the patient that indicates whether the patient had sepsis. Therefore, sepsis data is historic information associated with the patient where presence or absence of sepsis in the patient has already been determined. In an embodiment, the sepsis data may be a record of manual analysis of medical parameters associated with the patient that may be performed by a medical expert. Thus, the sepsis data associated with the patient is a benchmark based on which the output parameters of the first machine learning model may be baselined.

At step 306, a comparison is made between the first output parameter and the sepsis data. The comparison yields a presence or absence of difference between the sepsis data and the two output parameters. At step 307, it is determined if the first machine learning model is to be adjusted based on an outcome of the comparison. If there exists a need to adjust the first machine learning model, an adjustment is performed at step 308. In an embodiment, adjusting the first machine learning model may include updating one or more weights and biases associated with the first machine learning model.

In an instance where it is determined at step 302 that the medical dataset includes at least one sepsis specific parameter, a second output parameter is determined using a second machine learning model, at step 309. The second output parameter is a risk score and an indication of onset of sepsis in the patient. Further, at step 310, the second output parameter is compared with the sepsis data associated with the patient. At step 311, it is determined if the second machine learning model is to be adjusted based on the comparison between the second output parameter and the sepsis data. If a deviation is identified, the second machine learning model is adjusted, at step 312. Advantageously, adjusting enables improving the precision of the first machine learning model and the second machine learning models in determining the preliminary output parameter, the first output parameter and the second output parameter.

FIG. 4 illustrates a flowchart of a method 400 of pre-processing the medical dataset, according to an embodiment. The medical dataset is a training dataset received from various sources such as medical centers, etc. In the present embodiment, the medical dataset is labelled based on Sepsis-2 criteria and only the datasets that are ‘sepsis positive’ or ‘sepsis negative’ as per Sepsis-2 and Sepsis-3 criteria are considered for training the first machine learning model and the second machine learning model. Further, the medical dataset may be filtered based on criteria such as age and duration of patient's stay in an intensive care unit (ICU). Therefore, the medical dataset includes information associated with patients with more than 18 years of age and a length of stay in an ICU of more than 8 hours. At step 401, the medical dataset is imputed such that missing values in the medical dataset are eliminated/reduced. Imputation is performed for values associated with medical parameters in the medical dataset. For example, a time-limited, parameter-specific sample-and-hold-approach is used for performing the imputation. Therefore, if a missing value is identified in the medical dataset, the last available recording of the medical parameter value is carried forward for a limited time, depending on the type of medical parameter that is to be imputed. The time threshold may vary depending on the features in the medical dataset. For example, blood analyte levels associated with the patient are valid only in a time range of 22 to 24 hours. Therefore, if the determined substitution value lies outside of the pre-determined threshold, the imputation is not performed, and the value is labelled as missing. However, if the substitution value lies within the pre-determined threshold, the missing value in the medical dataset is replaced with the preceding value.

The first and the second machine learning models are trained on six laboratory parameters and age of the patient. The selected medical parameters represent different organs of the patient, thereby capturing signs of failure of these organs. The six laboratory parameters include blood urea nitrogen (BUN), creatinine, lactate/lactic acid, bilirubin total, WBCs, and platelets. In the present embodiment, the sepsis specific parameter considered is lactate/lactic acid. However, this can be replaced by another sepsis specific parameter including, but not limited to, IL-6, procalcitonin, and CRP. In an embodiment, the medical dataset may include more than sepsis specific parameter.

At step 403, the medical dataset is sampled/classified based on whether the medical dataset is associated with a ‘sepsis positive’ patient or a ‘sepsis negative’ patient. If the medical dataset is classified to be ‘sepsis positive,’ at step 404, a pre-onset and a post-onset time period from the medical dataset. Since, in a medical emergency department, only a single value associated with a medical parameter may be available (for a single time-instance), the first and the second machine learning models are trained using the most meaningful medical information from the medical dataset. Therefore, this enables the disclosure to function even in the absence of a time-series based information associated with the medical dataset. As illustrated in a graphical representation 500 in FIG. 5, in the present embodiment, the pre-onset time period 501 and post-onset time period 502 for medical dataset associated with ‘sepsis positive’ patients are determined as six hours each, before and after onset of sepsis in the patient.

Further, at step 405, at least one data point is extracted from the medical dataset such that the data point belongs to an earliest time instance in the pre-onset and/or post-onset time period. The data point corresponds to the medical parameters associated with the patient for that particular time instance and includes medical parameter values for at least four medical parameters associated with the patient. For example, if the selected data point has missing values for more than two medical parameters, the data point for the next hour is chosen. If the medical dataset is classified as ‘sepsis negative,’ at step 403, at least one data point is extracted from the medical dataset, at step 406. Sampling of data points associated with ‘sepsis negative’ medical datasets may be performed using the length of duration of stay of the patient(s) in the ICU, i.e., the time periods for onset of sepsis in ‘sepsis positive’ patients is analyzed, and the median may be used to sample data points associated with ‘sepsis negative’ medical datasets.

For the purposes to training the machine learning models, medical datasets associated with ‘sepsis negative’ patients are under-sampled to match the number of medical datasets associated with ‘sepsis positive’ patients. This enables reduction of a class imbalance between ‘sepsis positive’ and ‘sepsis negative’ patients due to higher number of medical datasets associated with ‘sepsis negative’ patients. In a further embodiment, if two data points are extracted from ‘sepsis positive’ medical datasets (one each from pre-onset and post-onset time periods), then the ‘sepsis negative’ medical datasets may be double in number than the ‘sepsis positive’ medical datasets.

In a further embodiment, one or more medical parameters may be present more often in a medical dataset associated with a ‘sepsis positive’ patient. In order to avoid correlation of these parameters with being sepsis positive, under-sampling may be performed such that the one or more medical parameters are present in equal proportions in both medical datasets associated with ‘sepsis positive’ and ‘sepsis negative’ patients. This enables elimination of undesirable biases in the machine learning models.

FIG. 6 illustrates a working of the first machine learning model and the second machine learning model 600 for determining the onset of sepsis in the patient, according to an embodiment. The first and the second machine learning model 600 is an Extreme Gradient (XG) Boost classification model. XGBoost classification model is optimized distributed gradient boosting system. XGBoost is an iterative decision tree algorithm with multiple decision trees in sequential form. Weights are assigned to each individual variable fed to the decision tree D₁-Dₙ. Each decision tree D₁-Dₙ computes the error in the previous decision tree, i.e., each tree D₁-Dₙ learns from residuals from all previous trees. The predicted output of XGBoost model 600 is a sum of results of all the decision trees D₁-Dₙ. Mathematically, the model 600 may be represented as:

$${\hat{y}}_{i} = {\sum }_{k = 1}^{n} {f}_{k} [ {x}_{i} ] , {f}_{k} ∈ F$$

wherein n is the number of decision trees, f is the functional space of F, F is the space of classification/regression trees, fₖ corresponds to a tree, fₖ[xᵢ] corresponds to result of tree k, and ŷᵢ is predicted value of i^{th} instance of xᵢ.

The objective of XGBoost is:

$${obj}_{( θ ) = {\sum }_{i = 1}^{n} l ( {y}_{i} , \hat{y} i )} + {\sum }_{k = 1}^{K} Ω ( {f}_{k} )$$

wherein the first term is the loss function, and the second term is a regularization parameter. Regularization enables avoiding overfitting of the model 600.

The hyperparameters for the model 600 are determined using Bayesian optimization based on which maximum depth, type of booster and learning rate were optimized. In the present embodiment, the first machine learning model is trained using a learning rate of 0.08814 and a maximum depth of 10, whereas the second machine learning model is trained using a learning rate of 0.1879 and a maximum depth of 11. The models 600 use gbtree as a booster and mlogloss as an evaluation metric.

The performance of the model 600 is calculated in terms of accuracy, Area Under the Receiver Operating Curve (AUROC), sensitivity, specificity, F1-score, and a negative predictive value (NPV). In an embodiment, the performance of the first machine learning model and the second machine learning model is presented below:

| Performance | First machine learning | Second machine |
|---|---|---|
| parameters | model | learning model |
| AUROC | 0.835 | 0.875 |
| Accuracy | 0.766 | 0.812 |
| Sensitivity | 0.781 | 0.823 |
| Specificity | 0.75 | 0.801 |
| F1 Score | 0.766 | 0.812 |
| NPV | 0.775 | 0.820 |

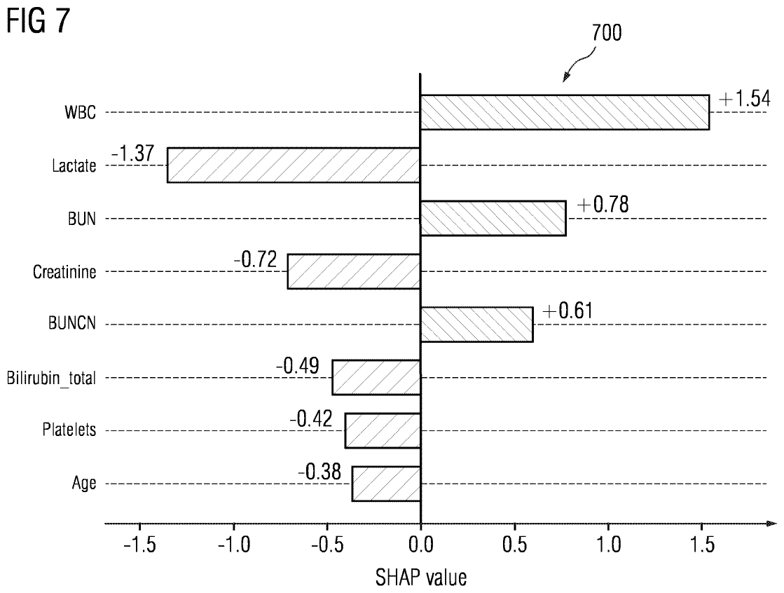

FIG. 7 illustrates a graphical representation 700 of root cause analysis of risk score determined by the first and the second machine learning models, according to an embodiment. In particular, the graphical representation illustrates contribution of individual medical parameters in the determination of onset of sepsis a patient, by the machine learning models. The model uses SHapley Additive explanations (SHAP) libraries to give real-time feature importance. For example, in the graphical representation 700, the Y-axis has medical parameters plotted in the decreasing order of importance while the SHAP values are plotted on the X-axis. Advantageously, this enables the user to determine a root cause of the risk score generated by the first and the second machine learning models.

The advantage of the disclosure is the method and device enable effective determination of onset of sepsis in a patient, in an emergency department set-up on a hospital/clinic. The disclosure analyzes the most commonly available laboratory parameters (WBC, creatinine, platelets, bilirubin and BUN) to predict whether sepsis-specific parameters are to be obtained for the patient. Such tests may be expensive and not routinely performed. Therefore, the disclosure provides a cost-effective and clinically adoptable solution to determine onset of sepsis in a patient. Furthermore, the performance of the second machine learning model improves significantly when a sepsis specific parameter associated with the patient is used in combination with the other commonly available medical parameters. This hierarchical approach adheres to the clinical feasibility where not all information might be available when a patient walks into the medical facility.

Additionally, the disclosure functions effectively even when minimal data points associated with the medical parameters are available, i.e., data points for single time instances enables effective determination of onset/presence of sepsis in the patient. Therefore, dependency on a time-series based information is reduced. Furthermore, the model is trained in a manner to avoid biases introduced by non-balanced number of medical parameters present in medical datasets associated with ‘sepsis positive’ and ‘sepsis negative’ patients. The foregoing examples have been provided merely for the purpose of explanation and are in no way to be construed as limiting of the present disclosure disclosed herein. While the disclosure has been described with reference to various embodiments, it is understood that the words, which have been used herein, are words of description and illustration, rather than words of limitation. Further, although the disclosure has been described herein with reference to particular means, materials, and embodiments, the disclosure is not intended to be limited to the particulars disclosed herein; rather, the disclosure extends to all functionally equivalent structures, methods and uses, such as are within the scope of the appended claims. Those skilled in the art, having the benefit of the teachings of this specification, may effect numerous modifications thereto and changes may be made without departing from the scope and spirit of the disclosure in its aspects.

1. A method of determining an onset of sepsis in a patient, the method comprising:
receiving a medical dataset associated with the patient, wherein the medical dataset comprises a plurality of medical parameters;
determining if the plurality of medical parameters includes at least one sepsis specific parameter;
determining, using a first trained machine learning model, a first output parameter, in response to the plurality of medical parameters not including the at least one sepsis specific parameter, wherein the first output parameter is a preliminary indication of onset of sepsis in the patient, and wherein the first output parameter is an indication of whether at least one sepsis specific parameter associated with the patient is to be obtained; and
determining, using a second trained machine learning model, a second output parameter in response to the plurality of medical parameters including the at least one sepsis specific parameter, the second output parameter being an indication of onset of sepsis in the patient,
wherein the plurality of medical parameters associated with the patient are obtained for at least one time instance.

2. The method according to claim 1, wherein, when the first output parameter indicates that the at least one sepsis specific parameter associated with the patient is to be obtained, the method comprises:
obtaining the at least one sepsis specific parameter associated with the patient;
providing the at least one sepsis specific parameter to the second trained machine learning model; and
determining, using the second trained machine learning model, the second output parameter.

3. The method according to claim 1, wherein the plurality of medical parameters comprises at least one of blood urea nitrogen, creatinine, bilirubin, white blood cells, platelets, lactate/lactic acid, C-reactive protein, procalcitonin, of IL-6.

4. The method according to claim 1, wherein the first output parameter is a risk score.

5. The method according to claim 4, wherein when the risk score is below a first pre-defined threshold, the first output parameter indicates no onset of sepsis in the patient,
when the risk score is above a second pre-defined threshold, the first output parameter indicates an onset of sepsis in the patient, and when the risk score is within a third pre-defined threshold range, at least one sepsis specific parameter associated with the patient is determined to be obtained.

6. The method according to claim 1, wherein the second output parameter is a risk score,
when the risk score is above a fourth pre-defined threshold, of the second output parameter indicates an onset of sepsis, and
the second output parameter has a greater accuracy in comparison to the first output parameter.

7. The method according to claim 1, wherein the first trained machine learning model and the second trained machine learning model are Extreme Gradient Boost classification models.

8. The method according to claim 1, further comprising:
determining a root cause of a risk score determined by the first trained machine learning model.

9. The method according to claim 1, further comprising:
determining a root cause of a risk score determined by the second trained machine learning model.

10. A method of training a first machine learning model and a second machine learning model for determining an onset of sepsis in a patient, the method comprising:
receiving a medical dataset associated with the patient, wherein the medical dataset comprises a plurality of medical parameters;
extracting the plurality of medical parameters from the medical dataset;
determining if the plurality of medical parameters comprises at least one sepsis specific parameter;
receiving the first machine learning model and the second machine learning model;
determining, by the first machine learning model, a first output parameter, the first output parameter being a preliminary indicator of onset of sepsis and whether the at least one sepsis specific parameter associated with the patient is to be obtained when the plurality of medical parameters does not comprise the at least one sepsis specific parameter;
receiving sepsis data related to the medical dataset, wherein the sepsis data indicates the onset of sepsis or indicates no presence of sepsis at a defined time period in the patient associated with the medical dataset;
adjusting the first machine learning model based on an outcome of a comparison between the first output parameter and the sepsis data;
determining, by the second machine learning model, a second output parameter, the second output parameter being an indicator of onset of sepsis in the patient when the plurality of medical parameters comprises the at least one sepsis specific parameter;
comparing the second output parameter with the sepsis data related to the medical dataset, wherein the sepsis data indicates the onset of sepsis or indicates no presence of sepsis at the defined time period in the patient associated with the medical dataset; and
adjusting the second machine learning model based on an outcome of a comparison between the second output parameter and the sepsis data.

11. The method according to claim 10, further comprising:
pre-processing the medical dataset related to the medical dataset.

12. The method according to claim 11, wherein the pre-processing of the medical dataset comprises:
imputing at least one missing value associated with the plurality of medical parameters in the medical dataset; and
sampling the plurality of medical parameters in the medical dataset based on a time instance associated with each medical parameter in the plurality of medical parameters.

13. The method according to claim 12, wherein the sampling of the plurality of medical parameters comprises:
identifying a pre-onset and post-onset time period from the medical dataset, wherein the medical dataset is associated with the patient identified as sepsis positive; and
extracting at least one data point from the medical dataset, wherein the data point is extracted from an earliest time instance in at least one of the pre-onset time period or the post-onset time period,
wherein the data point corresponds to the plurality of medical parameters.

14. The method according to claim 12, wherein the sampling of the plurality of medical parameters further comprises extracting at least one data point from the medical dataset, and
wherein the medical dataset is associated with the patient identified as sepsis negative.

15. A sepsis determination device for determining an onset of sepsis in a patient, the device comprising:
one or more processing units;
a medical database coupled to the one or more processing units, the medical database comprising a plurality of medical datasets associated with the patient and sepsis data; and
a memory coupled to the one or more processing units, the memory comprising a sepsis determination module configured to perform the method of claim 1, using a trained first machine learning model and a trained second machine learning model.

16. A computer program product comprising machine readable instructions, that when executed by one or more processing units, cause the one or more processing units to perform method steps according to claim 1.

17. A non-transitory computer readable medium on which program code sections of a computer program are saved, the program code sections being loadable into and/or executable in a system to make the system execute the method steps according to claim 1 when the program code sections are executed in the system.

18. The method according to claim 4, further comprising:
determining a root cause of the risk score determined by the first trained machine learning model.

19. The method according to claim 6, further comprising:
determining a root cause of the risk score determined by the second trained machine learning model.
